(19) United States
(12) Patent Application Publication
(10) Pub. No.: US 2025/0186799 A1
(43) Pub. Date: Jun. 12, 2025

(54) MEDICAL PROBE FOR OPTOGENETICS WITH VCSELs

(71) Applicant: COMMISSARIAT A L’ENERGIE ATOMIQUE ET AUX ENERGIES ALTERNATIVES, PARIS (FR)

(72) Inventors: Alexandre GARREAU, MASSY (FR); Fabien SAUTER-STARACE, GRENOBLE (FR); Jean-François PARET, MASSY (FR); Marie Blandine MARTIN, PALAISEAU (FR)

(21) Appl. No.: 18/968,221

(22) Filed: Dec. 4, 2024

(30) Foreign Application Priority Data
Dec. 8, 2023 (FR) 2313839

(51) Int. Cl. A61N 5/06; A61B 17/00; A61B 18/00; A61N 5/067; H01S 5/0233; H01S 5/042; H01S 5/42

(52) U.S. Cl. CPC A61N 5/0613; A61N 5/0601; A61N 5/067; H01S 5/0233; H01S 5/0421; H01S 5/423; A61B 2017/00526; A61B 2018/00083; A61N 2005/0668

(57) ABSTRACT

A medical probe for optogenetics, including a flexible substrate made of two-dimensional conductive material, a plurality of vertical-cavity surface-emitting III-V semiconductor microlasers, referred to as elementary lasers, including an active layer disposed between a lower reflective layer and an upper reflective layer, a lower semiconductor contact disposed between the lower reflective layer and the substrate, and a lower metal contact disposed on the substrate and connected to the lower semiconductor contact via the substrate, an upper semiconductor contact disposed on the upper reflective layer, and an upper metal contact connected to the upper semiconductor contact, the lower metal contacts of the elementary lasers being intended to be electrically connected to a common potential, a biocompatible encapsulation layer.

### CROSS-REFERENCE TO RELATED APPLICATION

This application claims priority to foreign French patent application No. FR 2313839, filed on Dec. 8, 2023, the disclosure of which is incorporated by reference in its entirety.

### FIELD OF THE INVENTION

The present invention relates to the field of optogenetics and more particularly to that of the medical probes for the implementation thereof.

### BACKGROUND

Optogenetics is a technique that involves genetically modifying neurons or nerves, such as the auditory nerve, so that they become sensitive to light through the expression of a protein: opsin.

An example of an application is to improve hearing.

A classic solution is to produce cochlear implants (CI), which are considered to be the best-performing neuroprosthesis that allows patients with sensory hearing loss to understand speech. By electrically stimulating the auditory nerve, cochlear implants provide an interface that reconnects the patient's brain to the auditory surroundings. However, since it is difficult to concentrate electrical current in conductive environments such as the cochlea, the accuracy of the electrical sound coding and the quality of artificial hearing are limited.

The principle of hearing is briefly outlined. The sound wave represents an alternation of high and low pressure zones. The tympanic membrane vibrates in response to a sound wave. This vibration is amplified as it passes through the ossicles (hammer, anvil and stirrup).

The amplified vibration is picked up by the oval window, causing pressure waves in the fluid of the scala vestibuli and scala tympani of the cochlea, the length of which is approximately 34 mm. The function of the cochlea is to map the sounds of different frequencies to corresponding characteristic positions of the basilar membrane, as is illustrated in FIG. 1.

Stereocils are actin-based protuberances on the auditory and vestibular sensory cells that are necessary for hearing. They convert the physical force of sound into an electrical signal by mechanical-electrical transduction. Defective stereocil homeostasis is one of the leading causes of progressive age-related deafness.

Optogenetic stimulation of the cochlea is an interesting alternative approach for the restoration of hearing. Cochlear optogenetics promises better spectral selectivity of artificial sound coding. It is based on the use of opsins injected into the cochlear branch of cranial nerve VIII, corresponding to the vestibulocochlear nerve, and an optical device stimulating the opsins. Opsins are light-sensitive proteins that convert a photon into an electrochemical signal.

The sensitivity curve of this opsin as a function of the wavelength of the light excitation is given, for example, by the publication by Klapoetke et al. “Independent optical excitation of distinct neural populations” Nat. Methods 11, 338-346 (2014). The normalized cumulative charge (NCC) as a function of wavelength λ, which reflects opsin sensitivity, is illustrated in FIG. 2 for different opsin types: Chrimson; VChR1; Chronos; ChR2; TsChR. For example, for Chrimson and its variants, the maximum sensitivity is at a wavelength of the order of 594 nm, and for TsChR the maximum sensitivity is at about 440 nm.

The publication by Keppeler et al “Multichannel optogenetic stimulation of the auditory pathway using microfabricated LED cochlear implants in rodents” Science Translational Medicine 12 (2020) describes an optical cochlear implant 35 (optogenetic) consisting of a flexible array of micro-LEDs based on gallium nitride (GaN), used for an application in a mouse model, and illustrated in FIG. 3. The LEDs 30 of 50×50 λm² are disposed on a polyimide substrate SubP, at one of its ends 31, at a pitch of 350 um. The part on which the LEDs 30 are disposed is intended to be inserted into the cochlear canal, and the substrate must therefore be very flexible. The LEDs are powered via electrical tracks connected at the other end to wires 32 which are connected to a connection interface 33. Each LED is powered via two contacts, a contact n and a contact p, respectively connected to the electrical tracks 34n and 34p, the contact n being common to all the LEDs according to a single track 34p. The substrate/LEDs assembly is encapsulated in a silicone layer 36 (see B in FIG. 3).

The fabrication of highly flexible polyimide devices having a thickness of 15 μm is made possible by a process of laser transfer of GaN-on-sapphire LEDs to a polyimide-on-silicon support wafer. With this transfer process, the LEDs are positioned one by one on the substrate, which makes the manufacture of the implant lengthy and expensive and limits the number of emitters of the device. Moreover, the large surface area of GaN-LEDs induces a large beam profile, which limits the number of optical emitters because of a risk of interaction between two neighbouring optical emitters. In addition, the large surface area of GaN-LEDs increases the temperature inside the cochlea, which requires that solutions be found to evacuate the heat.

An alternative solution based on microOLEDs is described in the publication by Sheppard et al. “Optogenetic stimulation probes with single-neuro resolution based on organic LED monolithically integrated on CMOS” Nature Electronics, vol. 6,pages 669-679 (2023). The device is illustrated in FIG. 4. The design and characterization of a silicon rod incorporating a high density of organic light sources. These microOLEDs, measuring 20 μm×20 μm with a pitch of 25 82 m, have the advantage of being processed after the electronic driver. It is what is called an “above-IC” device, which means that the deposits and structuring are effected after the chip (IC) production, on top, because the optical power of the microOLEDs is low. Although they are not very luminous, it is acknowledged that they can trigger an action potential associated with the opsin ChRmine, which is two orders of magnitude more sensitive than the opsin ChrimsonR, described by the abovementioned publication of Klapoetke et al, and sensitive to orange-coloured optical stimulation.

As before, a major drawback of these emitters is their poor efficiency, which induces a transformation of the supply current into heat by a Joule effect. However, medical standards do not allow tissue heating to exceed 2° C., which limits the number of transmitters that can be used. OLEDs are also sensitive to moisture, which leads to the use of a stack of atomic layers (atomic layer deposition (ALD)) and parylene, in order to ensure biocompatibility and limit moisture migration. The compatibility of these components with long-term use is not acquired.

### SUMMARY OF THE INVENTION

An object of the present invention is to remedy the aforementioned disadvantages by proposing a medical probe for optogenetics, which has emitters of small size and with better performance than the optogenetic implants of the prior art, and also a parallel manufacturing method.

The present invention relates to a medical probe for optogenetics, comprising:
a flexible substrate made of two-dimensional conductive material,
a plurality of vertical-cavity surface-emitting III-V semiconductor microlasers, referred to as elementary lasers, the elementary lasers being disposed on said substrate and integrated in an insulating layer, the elementary lasers having a maximum dimension of between 5 and 50 μm and comprising:
   an active layer disposed between a lower reflective layer and an upper reflective layer,
   a lower semiconductor contact disposed between the lower reflective layer and the substrate, and a lower metal contact disposed on the substrate and connected to said lower semiconductor contact via said substrate,
   an upper semiconductor contact disposed on the upper reflective layer, and an upper metal contact connected to said upper semiconductor contact,
   the lower metal contacts of the elementary lasers being intended to be electrically connected to a common potential,
a biocompatible encapsulation layer.

According to a first embodiment, the two-dimensional material is graphene.

According to a second embodiment, the two-dimensional material is a dichalcogenide or trichalcogenide configured to be conductive.

According to one embodiment, the substrate has the form of a ribbon, on a part of which said plurality of elementary lasers are disposed in a row, and the lower metal contacts of the elementary lasers are connected to a lower electrical track common to said elementary lasers of the row.

According to another embodiment, said plurality of elementary lasers are disposed in a matrix, the lower semiconductor contacts of the elementary lasers of a row of the matrix being connected to a lower electrical track common to the elementary lasers of said row of the matrix, the lower electrical tracks associated with the rows being connected to each other.

According to a first variant, the lower and upper semiconductor contacts of an elementary laser are made of gallium nitride or of a ternary material comprising gallium nitride.

According to an embodiment of the preceding first variant, the two-dimensional material is graphene and the lower semiconductor contact has a crystallographic growth axis along the axis.

According to another embodiment of the first variant, the two-dimensional material is a dichalcogenide chosen from WS₂, MoS₂, ReS₂, and the lower semiconductor contact has a crystallographic growth axis along the [100] axis.

According to a second variant, the lower and upper semiconductor contacts of an elementary laser are made of gallium arsenide or of a ternary material comprising gallium arsenide.

According to an embodiment of the second variant, the two-dimensional material is graphene, and the graphene substrate comprises seats in which the elementary lasers are disposed.

According to one embodiment, the active layer comprises multiple quantum wells or quantum dots.

According to one embodiment, the substrate made of two-dimensional material and the elementary lasers form a first structure, the probe comprising at least one second structure stacked on the first structure, elementary lasers of the two structures being arranged so that elementary lasers of the second structure do not mask a beam emitted by elementary lasers of the first structure.

According to one embodiment, the elementary lasers in the second structure are configured to emit a wavelength different from an emission wavelength of the first structure.

According to one embodiment, one of the two structures consists of elementary lasers comprising at least one layer of gallium nitride or a ternary material comprising gallium nitride, and the other structure consists of elementary lasers comprising at least one layer of gallium arsenide or a ternary material comprising gallium nitride.

According to another aspect, the invention relates to a first method for manufacturing a medical probe for optogenetics, comprising the steps of:
A1 making available a first initial substrate comprising a semiconductor substrate, an insulating layer substrate, a metal layer, and a graphene layer disposed on the metal layer,
B1 depositing on the first initial substrate a first dielectric layer and a first resin layer, structuring the first resin layer so as to form a first mask having first openings, the first openings having a maximum dimension of between 5 and 50 μm,
C1 etching the first dielectric layer as far as the graphene layer by wet etching, so as to expose the graphene layer in the first openings,
D1 removing the first resin layer,
E1 producing a stack of semiconductor materials in said first openings by epitaxy, the stack comprising a lower semiconductor contact of gallium nitride grown by epitaxy on the graphene layer, a lower reflective layer, an active layer, an upper reflective layer, and an upper semiconductor contact made of gallium nitride,
F1 depositing a second dielectric layer, structuring a second resin layer so as to form a second mask having second openings above each stack, etching the second dielectric layer and the upper semiconductor contact, and removing the second resin layer,
G1 removing the first and second dielectric layers by wet etching, so as to obtain stacks disposed on the first initial substrate,
H1 depositing a lower metal contact on the graphene layer, depositing an insulating layer around the stacks, and depositing an upper metal contact connected to the upper semiconductor contact,
I1 removing the semiconductor substrate using the insulating layer substrate,
J1 removing the metal layer.

In addition, the stacks and the associated first and second metal contacts are configured to form vertical-cavity surface-emitting semiconductor microlasers inserted in the insulating layer, referred to as elementary lasers, the graphene layer forming a flexible substrate, the elementary lasers being disposed on said flexible substrate. The method further comprises a step K1 of encapsulating the substrate and the elementary lasers with a biocompatible material.

According to one embodiment, during step E1, the epitaxial growth of the lower gallium nitride semiconductor contact on the graphene substrate takes place in a direction [1000].

According to one embodiment, in step C1, the wet etching of the first dielectric layer as far as the graphene layer is of the buffered oxide etching (BOE) type.

According to another aspect, the invention relates to a second method for manufacturing a medical probe for optogenetics, comprising the steps of:
A2 making available a second initial substrate comprising a gallium arsenide substrate, an insulating layer substrate, a metal layer, and a first graphene layer disposed on the metal layer,
B2 depositing on the second initial substrate a first dielectric layer and a first resin layer, structuring the first resin layer so as to form a first mask having first openings, the first openings having a maximum dimension of between 5 and 50 μm,
C2 etching the first dielectric layer as far as the graphene layer,
D2 removing the first resin layer, etching the graphene layer, etching the metal layer and etching the insulating layer substrate, so as to expose the gallium arsenide substrate in the first openings,
E2 producing by epitaxy, in said first openings, a stack of semiconductor materials comprising a lower semiconductor contact of gallium arsenide epitaxially grown on the gallium arsenide substrate, a lower reflective layer, an active layer, an upper reflective layer, and an upper semiconductor contact made of gallium arsenide,
F2 depositing a second dielectric layer and a second resin layer, structuring the second resin layer so as to form a second mask having second openings above each stack, etching the second dielectric layer, etching the upper semiconductor contact, and removing the second resin layer,
G2 removing the first and second dielectric layers by wet etching so as to obtain stacks disposed on the gallium arsenide substrate,
H2 depositing a lower metal contact on the remaining graphene layer on each side of the stacks, depositing an insulating layer around the stacks, and depositing an upper metal contact in contact with the upper semiconductor contact,
I2 removing the gallium arsenide substrate using the insulating layer substrate,
J2 removing the metal layer, and depositing a second graphene layer on the first graphene layer and the lower semiconductor contact, the first and second graphene layers collectively forming a flexible graphene substrate.

In addition, the stacks and the associated first and second metal contacts are configured to form vertical-cavity surface-emitting semiconductor microlasers (μVL) inserted in the insulating layer, referred to as elementary lasers, the elementary lasers being disposed on said graphene substrate, the graphene substrate comprising seats in which the elementary lasers are disposed. In addition, the method comprises a step K2 of encapsulating the substrate and the elementary lasers with a biocompatible material.

The following description presents several exemplary embodiments of the device of the invention: these examples do not limit the scope of the invention. These exemplary embodiments contain not only the features essential to the invention but also additional features associated with the embodiments in question.

### BRIEF DESCRIPTION OF THE DRAWINGS

The invention will be better understood and other features, aims and advantages thereof will become apparent from the following detailed description, which is provided with reference to the appended drawings, which are given by way of non-limiting examples and in which:

FIG. 1, already cited, is a diagram illustrating the principle of sound transmission by the cochlea.

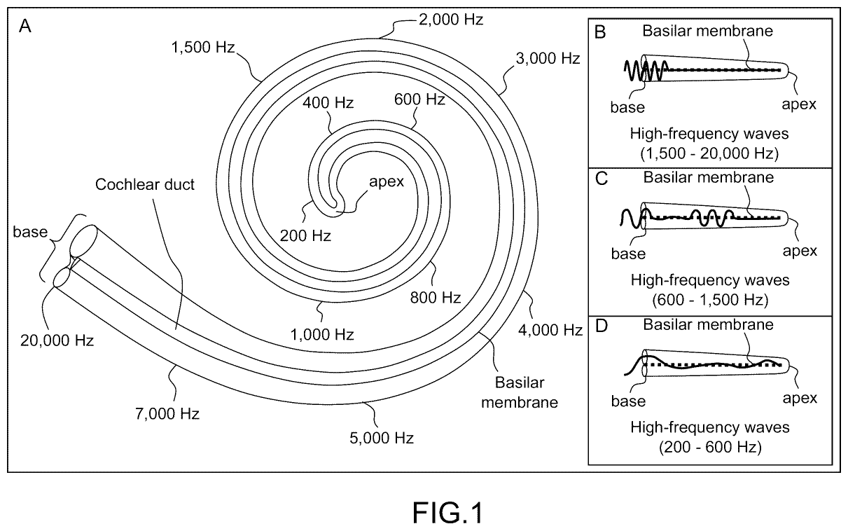

FIG. 2, already cited, illustrates the cumulative charge normalized as a function of wavelength, for different types of opsin.

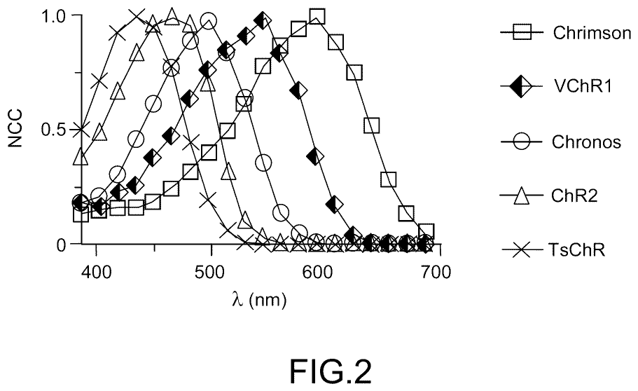

FIG. 3, already cited, illustrates an optical cochlear implant consisting of a flexible array of gallium nitride-based micro-LEDs for a mouse.

FIG. 4, already cited, illustrates an implant consisting of microOLEDs on CMOS.

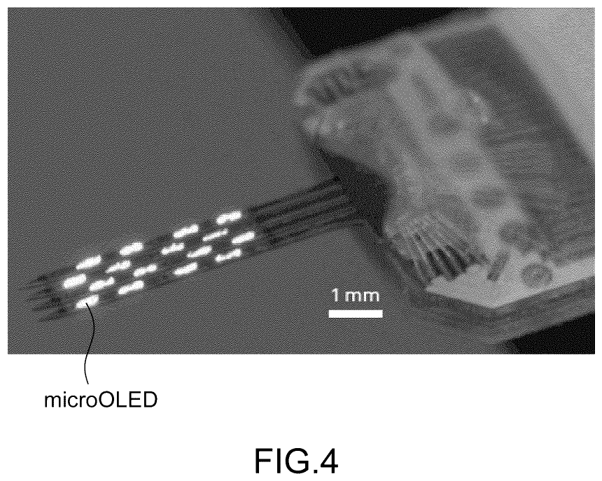

FIG. 5 illustrates an embodiment of a probe according to the invention in which the substrate is in the form of a ribbon.

FIG. 6 illustrates an embodiment of a probe according to the invention in which the elementary lasers are disposed according to a planar array in a matrix.

FIG. 7 illustrates an embodiment of a probe according to the invention in which the graphene substrate comprises seats in which the GaAs-based elementary lasers are disposed.

FIG. 8 illustrates an embodiment of a probe according to the invention in which the substrate made of 2D material and the elementary lasers form a first structure S1, and the probe comprises at least one second structure S2 stacked on the first structure S1, the structure S2 having an architecture identical to the structure S1 (substrate+elementary lasers).

FIG. 9 illustrates steps A1 to F1 of the method 100 according to the invention.

FIG. 10 illustrates steps G1 to J1 of the method 100 according to the invention.

FIG. 11 illustrates steps A2 to F2 of the method 100 according to the invention.

FIG. 12 illustrates steps G2 to J2 of the method 100 according to the invention.

### DETAILED DESCRIPTION

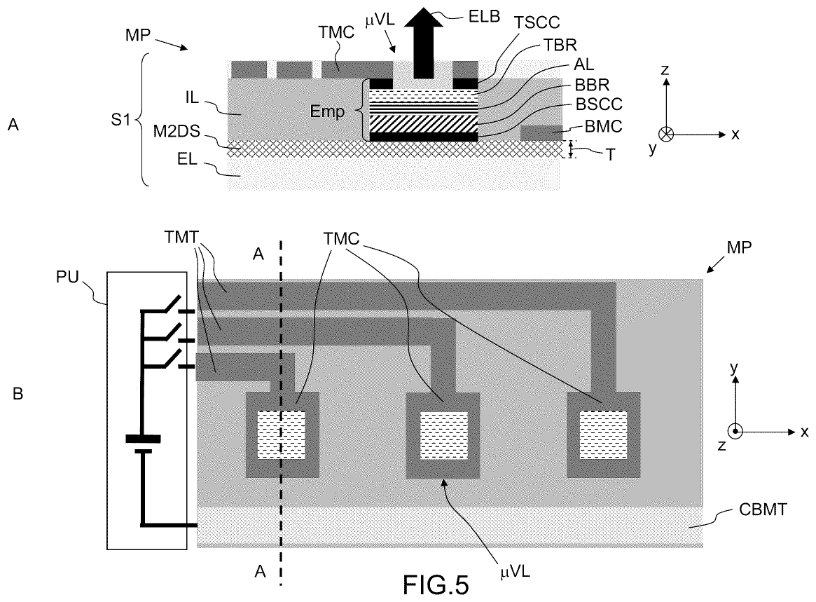

An embodiment of the medical probe MP according to the invention, adapted for the production of a cochlear implant with a substrate of ribbon form and in-line emitters, is illustrated in FIG. 5. Part B is a top view, in an XY plane, of the probe according to the invention, and part A is a profile view in an XZ plane (section along AA).

The design of the cochlear implant is facilitated by the fact that the topology of the cochlea is well known, with its distribution of hair cells specific to the sound frequencies. After recording and analysing the frequency of an emitted sound, an emitter of the optical probe, the location of which in the cochlea corresponds to this frequency, is switched on. It makes it possible to stimulate the cochlear branch of cranial nerve VIII, into which opsins have been injected, and thus generate artificial sound perception.

However, the principle of stimulation of a neuron or nerve by an optical device via the opsin is applicable to any other application in optogenetics, by modifying the design of the device. The invention is thus applicable to other types of optogenetic probes having different emitter arrangement geometries, for example a planar array, a rod, a tube obtained from a planar array (by virtue of the flexible substrate), etc. The probe according to the invention can be applied, for example, for integration into the visual cortex for restoring sight or into the motor cortex for compensation of motor handicaps.

The medical probe MP for optogenetics according to the invention comprises a flexible substrate M2DS made of two-dimensional (2D) material and configured to be conductive.

The 2D materials have a planar structure and are composed of one to a few monolayers L, each monolayer comprising a few atomic planes (typically 1 to 20), the number of atomic planes being a function of the atomic structure. The chemical bonds within a monolayer are covalent. A two-dimensional material can be conductive or semiconductive, typically depending on the number of stacked monolayers.

According to one embodiment, the two-dimensional material is graphene. Graphene is naturally conductive and consists of a single atom, carbon, and the monolayer comprises only one atomic plane; it is planar and consists of carbon atoms arranged in the form of a hexagonal lattice. Graphene is flexible, and the thickness T of such a substrate is typically between 0.3 and 20 nm depending on the number of stacked monolayers. The advantages of graphene are that the associated methods are fully developed and that it is non-toxic. It also has good lattice matching with certain III-V semiconductors (see below).

According to another embodiment, the two-dimensional material is a dichalcogenide or a trichalcogenide configured to be conductive, having a thickness of between 0.3 and 20 nm.

The probe MP also comprises a plurality of vertical-cavity surface-emitting III-V semiconductor microlasers μVL, referred to as elementary lasers, conventionally designated as VCSELs, standing for vertical-cavity surface-emitting lasers. The elementary lasers μVL are disposed on the substrate M2DS and integrated in an insulating layer IL. The elementary lasers have a maximum dimension of between 5 and 50 μm. The maximum dimension is understood to mean the largest lateral dimension of the laser. Typically, lasers have a thickness (height) of the order of 0.5 to 5 μm.

An elementary laser of the probe according to the invention has a conventional layer structure and comprises an active layer AL disposed between a lower reflective layer BBR and an upper reflective layer TBR. It also comprises:
a lower semiconductor contact BSCC disposed between the lower reflective layer and the substrate, and a lower metal contact BMC,
an upper semiconductor contact TSCC disposed on the upper reflective layer TBR, and an upper metal contact TMC connected to the upper semiconductor contact.

In the configuration of the invention, the lower metal contact is disposed on the substrate M2DS and electrically connected to the lower semiconductor contact BSCC via the substrate, which is conductive. However, it is expedient that the electrical connectivity is sufficient such that the lower metal contact is not too far away from the lower semiconductor contact.

Furthermore, the lower metal contacts of the elementary lasers are intended to be electrically connected to a common potential, typically a reference potential, or a ground.

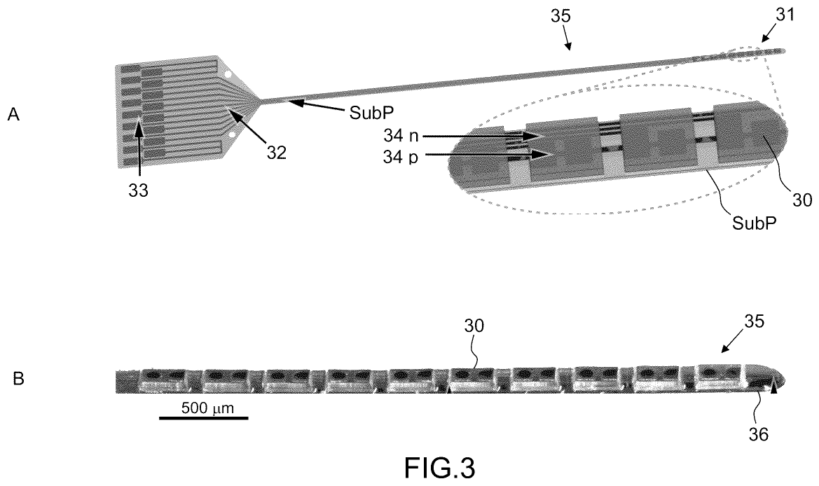

Conventionally, for a probe having several emitters (see FIG. 3 for example), the lower and upper metal contacts are connected to associated electrical tracks, typically metal, which convey the electrical signal controlling the emitters.

The medical probe MP according to the invention is intended to be electrically connected to a power supply and control unit PU, connected to the tracks.

For elementary lasers of the VCSEL type according to the invention, the lower metal contacts are electrically connected to each other via the conductive substrate and are intended to be connected to the common potential typically via tracks connected, at the end of the probe, to the power supply and control unit.

Preferably, the lower semiconductor contacts are connected to one or more lower electrical tracks CMT (a function of the arrangement of the elementary lasers) disposed on the substrate.

Despite the conductive nature of the substrate, the lower metal contacts and the lower electrical tracks are necessary to transport the current flowing in the elementary lasers. There is typically a factor of at least 4 to 5 between the conductivity of a metal (gold, copper) and that of a 2D material.

The upper metal contacts are connected to upper electrical tracks TMT. For each laser, the upper electrical contact TMC is connected to an upper track TMT associated with the laser which conveys the elementary laser control signal.

According to the embodiment of FIG. 5, the substrate is in the form of a ribbon, on a part of which lasers are arranged in a row. The lower metal contacts of the plurality of lasers are connected to a lower electrical track CBMT which is common to the elementary lasers of the row and is intended to be connected to the electrical ground of the device. This allows the number of electrical contacts to be halved. This geometry is well suited to a cochlear implant.

The probe MP also comprises a biocompatible, electrically insulating and transparent encapsulation layer having long-term stability, for example a medical-grade silicone, which surrounds at least that part of the probe intended to be inserted into the body.

The originality of the invention is that a medical probe for optogenetics, comprising VCSELs of small dimension, is realized on a graphene substrate that is thin and flexible, this structure having many advantages over existing probes.

First of all, the small size of the elementary lasers and the very directive nature of the emitted light beam ELB enables a very dense arrangement of the emitters on the substrate made of 2D material, with the limit of being able to pass the electrical wiring if necessary (typically the tracks are a few microns wide). For in-line lasers such as in part B of FIG. 5, the lasers can be very close together, because there are no tracks between two lasers. A dense arrangement allows the target cells to be addressed with very good spatial resolution.

In addition, VCSELs have a much better quantum efficiency than LEDs (more electrons transformed into photons), which entails a much lower generation of heat by a Joule effect. This is very important for medical probes, since the permitted heating of the device is very limited. It is therefore not necessary to provide an additional device for heat removal, and this also enables dense arrangement of the emitters without excessive heat emission.

Finally, the substrate made of 2D material confers great flexibility on the device, allowing it to adapt to different environments of the human body.

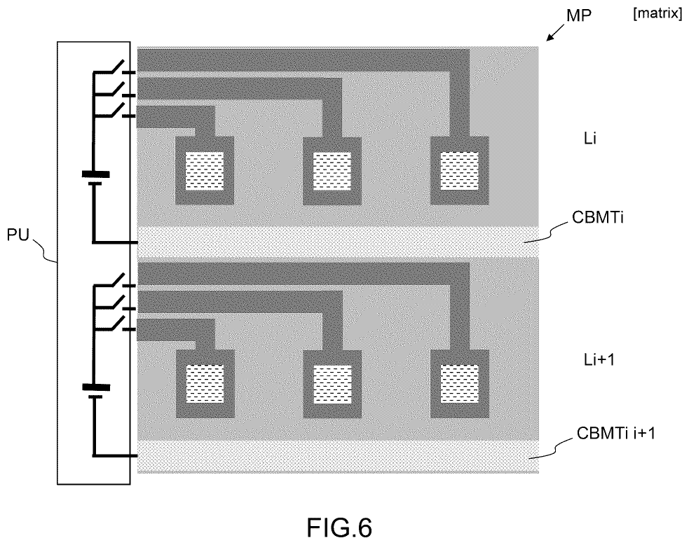

According to one embodiment, the elementary lasers are disposed according to a planar matrix array as illustrated in FIG. 6. Preferably, the elementary lasers of a row Li (index i) of the matrix are connected to an associated shared lower electrical track CBMTi.

The elementary laser μVL is a vertical-cavity surface-emitting laser based on a stack of III-V semiconductor layers. Preferably, it comprises a p-doped III-V contact, a p-doped III-V Bragg reflector, the active layer AL, an n-doped III-V Bragg reflector and a p-doped III-V contact.

According to one embodiment, the active layer AL of the laser contains multiple quantum wells MQW based on III-V semiconductors, whose structure is an alternating stack of wells and barriers composed of III-V semiconductors. According to another embodiment, the active layer of the laser contains quantum dots QD which are based on III-V semiconductors.

According to a first variant of the probe according to the invention, the lower semiconductor contacts BSCC and upper semiconductor contacts TSCC of an elementary laser are made of gallium nitride GaN or a ternary material comprising gallium nitride. This family of GaN-based VCSELs emits a light beam ELB having a wavelength in a range from blue to green.

According to a second variant of the probe according to the invention, the lower semiconductor contacts BSCC and upper semiconductor contacts TSCC of an elementary laser are made of gallium arsenide (GaAs) or a ternary material comprising gallium arsenide. This family of GaAs-based VCSELs emits a light beam ELB having a wavelength in a range from orange to red or infrared, depending on the nature of the active layer.

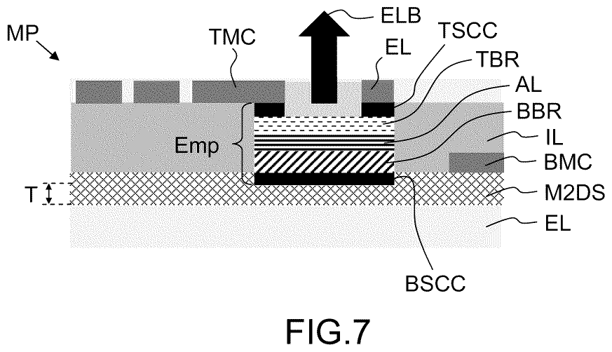

As will be seen later, the steps of the method for manufacturing elementary lasers on a substrate made of 2D material which is graphene differ for the two families of III-V semiconductor laser components. This means that the GaAs-based lasers μVL are indeed arranged on the graphene substrate, but the latter includes seats in which the elementary lasers are disposed, as is illustrated in FIG. 7.

The two VCSEL families make it possible to target two different wavelength ranges.

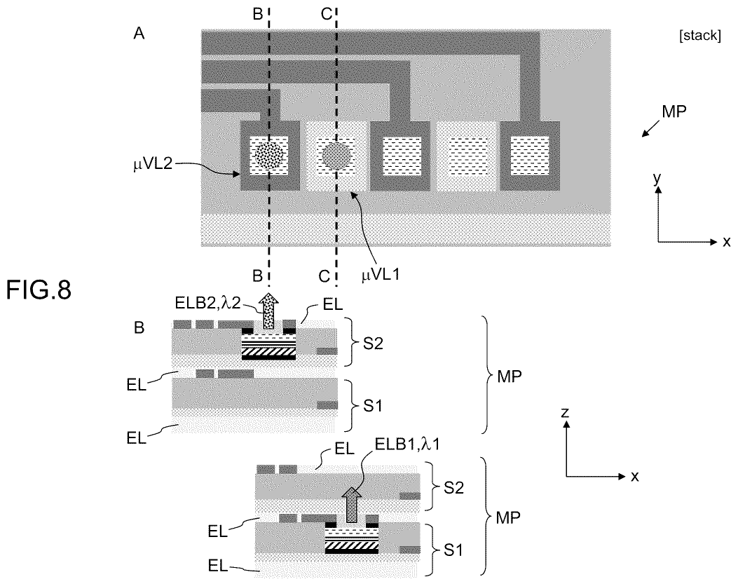

According to an embodiment illustrated in FIG. 8, it is considered that the substrate made of 2D material and the elementary lasers form a first structure S1, and the probe MP comprises at least one second structure S2 stacked on the first structure S1, the structure S2 having an architecture identical to the structure S1 (substrate+elementary lasers). It is then the assembly S1+S2 that is embedded in an encapsulation layer. Preferably, elementary lasers of the two structures are arranged such that elementary lasers μVL2 of the second structure do not mask a beam ELB1 emitted by elementary lasers μVL1 of the first structure, as is illustrated in part A of FIG. 8 (top view of the probe). Part B shows a profile view along the sections BB and CC.

Preferably, the elementary lasers in the second structure are configured to emit a wavelength λ2 different from an emission wavelength λ1 of the first structure. This makes it possible to have a probe that can be located near the cells of interest and can emit sometimes one colour sometimes another. Such a probe makes it possible, in combination with cells incorporating an excitatory opsin (for example at λ1) and an inhibitory opsin (for example at λ2), to excite and inhibit cell channels on command, by turning the associated dual-level emitters of the probe on and off.

According to an embodiment of the dual-level probe of FIG. 8, one of the two structures consists of elementary lasers comprising at least one layer of gallium nitride (GaN-based component family), and the other structure consists of elementary lasers comprising at least one layer of gallium arsenide (GaAs-based component family).

According to one embodiment of an emitter according to the first variant, the lower semiconductor contact BSCC is made of n-doped gallium nitride, and the upper semiconductor contact TSCC is made of p-doped gallium nitride. It is possible to reverse the dopings.

The lower and upper reflective layers BBR and TBR are, for example, alternating layers of AlGaN and GaN, and the active layer AL comprises multiple quantum wells of InGaN separated by gallium nitride barriers.

Typically, the lower semiconductor contact BSCC made of gallium nitride has a wurtzite crystallographic structure.

According to one embodiment of a probe according to the invention with emitters according to the first variant, the two-dimensional material is graphene and the lower semiconductor contact BSCC has a crystallographic growth axis along the

axis. For the realization of such an emitter on graphene, the lattice agreement between GaN and graphene favours such a structure.

Conventionally, when the lower semiconductor contact is along the axis and all the materials of the stack forming the VCSEL are GaN-based, these materials all have a growth axis along [1000].

According to another embodiment of an emitter according to the first variant, the two-dimensional material is a dichalcogenide or a trichalcogenide. Preferably, the material is a dichalcogenide chosen from WS₂, MoS₂, ReS₂, and the lower semiconductor contact has a crystallographic growth axis along the [100] axis. The aforementioned materials have the advantage of having an atomic lattice structure close to that of GaN, and the structure of GaN along the [100] axis is the most suitable for GaN growth on this type of material.

According to an embodiment of an emitter according to the second variant, the lower semiconductor contact BSCC and the upper semiconductor contact TSCC are made of GaAs or of a ternary material comprising GaAs.

Preferably, one of the semiconductor contacts is n-doped and the other is p-doped.

For example, the lower reflective layer BBR is an alternation of two layers of AlGaAs and AlAs, the active layer AL comprises multiple quantum wells of GalnP separated by AlGalnP barriers, and the upper reflective layer TBR is an alternation of two layers of AlGaAs of different composition.

According to another aspect, the invention relates to a method 100 for manufacturing a medical probe for optogenetics having elementary lasers made on the basis of GaN (at least the lower semiconductor contact) on a substrate made of 2D material of the graphene type. Graphene has the advantage of having a fully developed method of implementation and of being non-toxic. It also has the advantage of having a lattice structure in accordance with that of GaN.

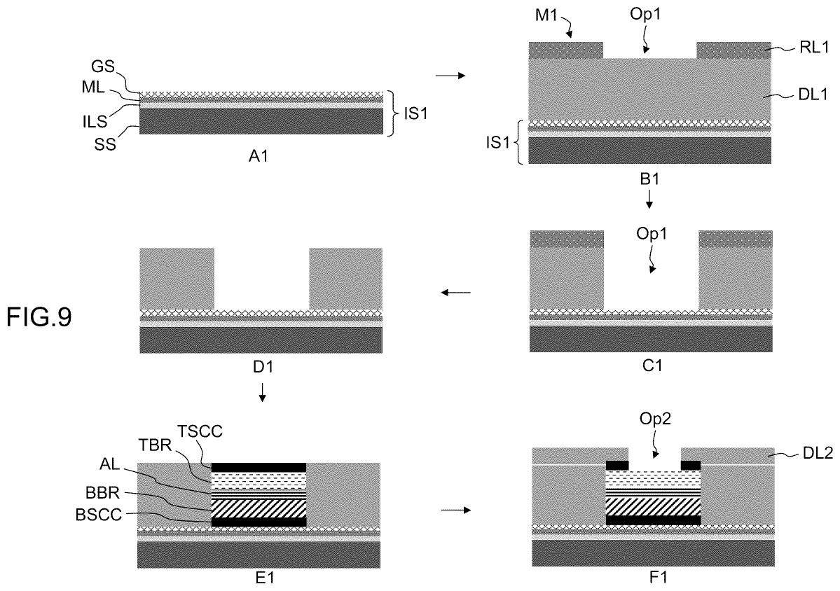

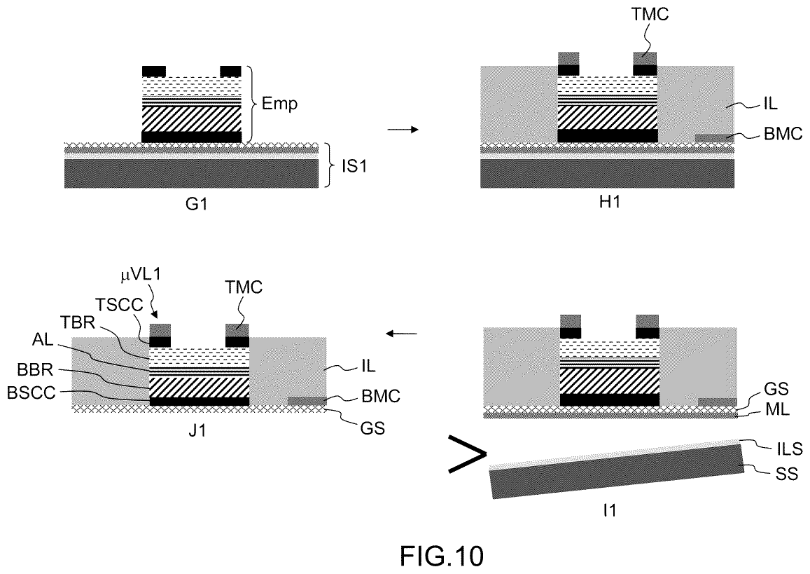

The method 100 according to the invention is illustrated in FIG. 9 for steps A1 to F1 and in FIG. 10 for steps G1 to J1.

It comprises a first step A1 consisting in making available a first initial substrate IS1 comprising a semiconductor substrate SS, such as silicon, an insulating layer substrate ILS, typically dielectric, a metal layer ML, for example of copper or nickel, and a graphene layer GS disposed on the metal layer. Graphene is not soluble in these metals. Typically, the graphene layer has grown on the metal layer by a technique of chemical vapour deposition (CVD) using CH₄-H₂.

Then, in a step B1, a first dielectric layer (DL1), typically SiO₂ or Si₃N₄, and a first resin layer RL1 are deposited on the first initial substrate IS1, and the first resin layer RL1 is structured, typically by photolithography, so as to form a first mask M1 having first openings Op1. The first openings have a maximum dimension of between 5 and 50 μm.

In a step C1, the first dielectric layer DL1 is etched as far as the graphene layer by wet etching, so as to expose the graphene layer in the first openings. One stops here at a layer of 2D material of atomic thickness, and the integrity of the graphene layer must be preserved. It is not possible to use a dry etching technique of the ion type (or reactive-ion etching (RIE)), for example based on chlorine, which is necessarily partly mechanical and could damage the graphene. Only wet etching can be used for this step. Preferably, according to one embodiment, a buffered hydrofluoric acid (HF) type etching is used, such as buffered oxide etching (BOE), since graphene resists this type of etching.

In a step D1, the first resin layer is removed, for example by chemical attack with 2-propanol.

One then has locations in which the VCSELs are grown. Thus, in a step E1, a stack of semiconductor materials is produced by epitaxy, typically by metal-organic chemical vapour deposition (MOCVD) or gas source molecular beam epitaxy (GSMBE) in the first openings Op1. The stack Emp comprises a lower semiconductor contact BSCC made of gallium nitride (or a GaN-based ternary) grown by epitaxy on the graphene layer, a lower reflective layer BBR, an active layer AL, an upper reflective layer TBR, and an upper semiconductor contact TSCC made of gallium nitride (or a GaN-based ternary).

According to a preferred embodiment, the GaN epitaxy on graphene is carried

out along the crystallographic axis [1000], because there is a good lattice agreement. Preferably, the lower semiconductor contact BSCC has a wurtzite crystallographic structure.

Thus, in the locations where the graphene is exposed, VCSELs are grown, all isolated from one another, with a common mass. By virtue of this technology called selective area growth (SAG), the semiconductor grows on the graphene and not elsewhere, which makes it possible to define the geometry of the laser without chlorinated etching (just etching of the dielectric layer and of the contact to allow the light out).

In a step F1, a second dielectric layer DL2 (for example of SiO₂ or Si₃N₄) is deposited, and a second resin layer RL2 is structured, typically by photolithography, so as to form a second mask M2 having second openings Op2 above each stack. The second dielectric layer DL2 is then etched, typically by fluorine RIE, and the upper semiconductor contact is etched, typically by chlorine RIE. Finally, the second resin layer RL2 is removed. The emission surface of the VCSEL is exposed for the passage of the emitted light. The graphene layer is protected by the layer DL2.

In step G1, the first and second dielectric layers DL1 and DL2 are removed by wet etching, so as to obtain stacks disposed on the first initial substrate. The etching here is wet etching, for the same reasons as before, i.e. so as not to damage the graphene layer.

Next, in step H1, a lower metal contact BMC is deposited on the graphene layer, which is thus connected to the lower semiconductor contact via the conductive graphene layer, and an insulating layer IL is deposited around the stacks. An upper metal contact TMC is also deposited, connected to the upper semiconductor contact TSCC, and such that emission can occur via the upper surface of the VCSEL. At this stage, the elementary lasers are finished. It is also at this stage that the one or more lower tracks CBMT and upper tracks TMT are made for the connections.

In a step I1, the semiconductor substrate SS is removed using the insulating layer substrate ILS, preferably by mechanical cleavage. The presence of the insulating layer substrate ILS helps the decoupling between the graphene layer and the metal layer ML.

Then, in a step J1, the metal layer ML is removed, for example by chemical attack based on FeCl₃ when the layer ML is made of copper.

The stacks and the associated first and second metal contacts are configured to form vertical-cavity surface-emitting semiconductor microlasers μVL inserted in the insulating layer, referred to as elementary lasers. The graphene layer forms a flexible substrate GS, the elementary lasers being disposed on this flexible substrate.

The method 100 further comprises a step K1 of encapsulating the substrate and the elementary lasers with a biocompatible material.

The method 100 according to the invention thus makes it possible to produce in parallel a large number of elementary lasers on a flexible substrate of graphene, all at once and without a transfer process.

The elementary lasers are all at the same ground potential on account of the conductivity of the graphene.

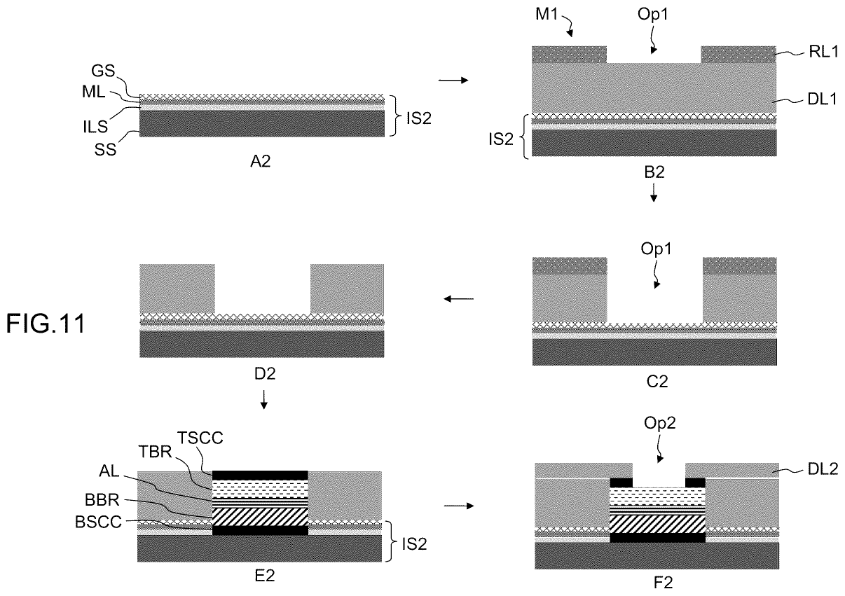

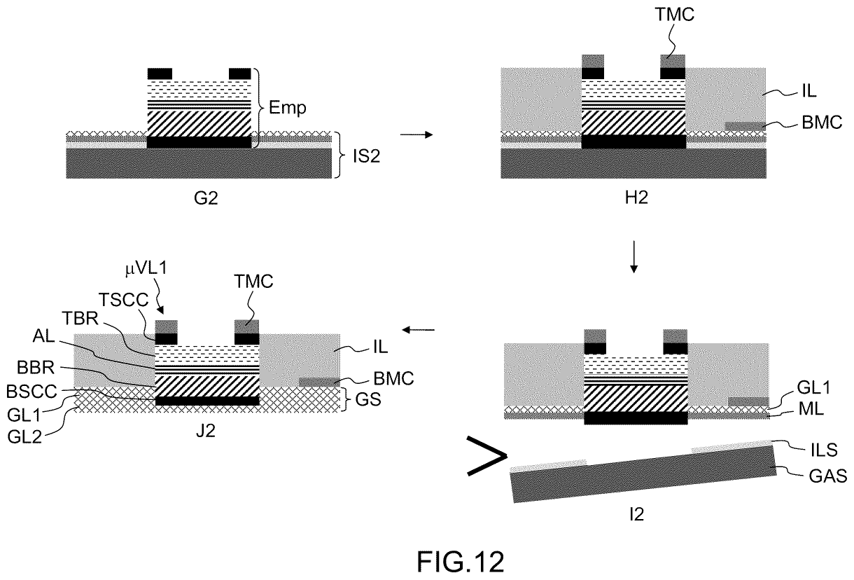

According to another aspect, the invention relates to a method 200 for manufacturing a medical probe for optogenetics having elementary lasers made on the basis of GaAs on a substrate made of 2D material of the graphene type. The method 200 according to the invention is illustrated in FIG. 11 for steps A2 to E2 and in FIG. 12 for steps G2 to J2.

The method 200 has some steps that are different from those of the method 100, because GaAs is not matched in lattice terms with the 2D materials such as graphene or dichalcogenides.

In a first step A2, a second initial substrate IS2 is provided, comprising a gallium arsenide substrate GAS, an insulating layer substrate ILS, a metal layer ML (for example of copper or nickel), and a first graphene layer GL1 disposed on the metal layer.

In a step B2, a first dielectric layer DL1 and a first resin layer RL1 are deposited on the second initial substrate, and the first resin layer is structured so as to form a first mask M1 having first openings Op1. The first openings have a maximum dimension of between 5 and 50 μm.

In a step C2, the first dielectric layer is etched as far as the graphene layer. The etching here can be dry or wet, because the graphene layer GL1 will be removed.

In a step D2, the first resin layer is removed, the graphene layer is etched, for example by RIE with chlorine, and the metal layer is etched, typically by ion beam etching (IBE). The insulating layer substrate is also etched, for example by RIE with fluorine, so as to expose the GaAs substrate in the first openings Op1.

Thus, in the locations where the substrate GAS is exposed, VCSELs are grown, all isolated from one another, typically with a common mass (see below). The GaAs material does not grow on graphene, because it is not adapted for the graphene lattice. In the method 200, which is still of the type with SAG in the openings Op1, the graphene becomes a growth mask.

In a step E2, a stack of semiconductor materials comprising a lower semiconductor contact BSCC made of gallium arsenide (or of a GaAs-based ternary material) epitaxially grown on the gallium arsenide substrate GAS, a lower reflective layer BBR, an active layer AL, an upper reflective layer TBR and an upper semiconductor contact TSCC of gallium arsenide, is formed by epitaxy, typically MOCVD or GSMBE, in the first openings Op1.

In the same way as in the method 100, in a step F2 a second dielectric layer DL2 and a second resin layer RL2 are deposited, the second resin layer RL2 is structured so as to form a second mask M2 having second openings Op2 above each stack, the second dielectric layer is etched, the upper semiconductor contact is etched, and the second resin layer is removed.

In a step G2, the first and second dielectric layers are removed by wet etching, so as to obtain stacks disposed on the gallium arsenide substrate GAS. Wet etching is used here so as not to damage the graphene remaining on each side of the stack.

In a step H2, the lower metal contact BMC is deposited on the remaining graphene layer on each side of the stacks, an insulating layer IL is deposited around the stacks and an upper metal contact TMC is deposited in contact with the upper semiconductor contact TSCC. It is also at this stage that the one or more lower tracks and upper tracks are made for the connections.

In a step I2, the gallium arsenide substrate GAS is removed using the insulating layer substrate ILS, preferably by mechanical cleavage.

In a step J2, the metal layer ML is removed and a second graphene layer GL2 is deposited on the first graphene layer GL1 and the lower semiconductor contact, the first and second graphene layers collectively forming a flexible graphene substrate GS.

This second layer of graphene ensures the solidity and flexibility of the graphene substrate and the injection of current. The stacks and the associated first and second metal contacts are configured to form vertical-cavity surface-emitting semiconductor microlasers μVL inserted in the insulating layer, referred to as elementary lasers. The elementary lasers are disposed on the graphene substrate GS, and here the graphene substrate comprises seats in which the elementary lasers are disposed.

The method 200 further comprises a step K2 of encapsulating the substrate and the elementary lasers with a biocompatible material.

Thus, in this method, the graphene is etched as far as the GaAs, the laser component is grown on a GaAs substrate GAS, and then the component is detached from the substrate GAS using the insulating layer substrate.

1. A medical probe (MP) for optogenetics, comprising:
a flexible substrate made of two-dimensional conductive material (M2DS), a plurality of vertical-cavity surface-emitting III-V semiconductor microlasers (μVL), referred to as elementary lasers, the elementary lasers being disposed on said substrate and integrated in an insulating layer (IL), the elementary lasers having a maximum dimension of between 5 and 50 μm and comprising:
an active layer (AL) disposed between a lower reflective layer (BBR) and an upper reflective layer (TBR),
a lower semiconductor contact (BSCC) disposed between the lower reflective layer and the substrate, and a lower metal contact (BMC) disposed on the substrate and connected to said lower semiconductor contact (BSCC) via said substrate (M2DS),
an upper semiconductor contact (TSCC) disposed on the upper reflective layer (TBR), and an upper metal contact (TMC) connected to said upper semiconductor contact,
the lower metal contacts of the elementary lasers being intended to be electrically connected to a common potential,
a biocompatible encapsulation layer.

2. The medical probe (MP) according to claim 1, wherein the two-dimensional material is graphene.

3. The medical probe (MP) according to claim 1, wherein the two-dimensional material is a dichalcogenide or trichalcogenide configured to be conductive.

4. The medical probe according to claim 1, wherein the substrate has the form of a ribbon, on a part of which said plurality of elementary lasers are disposed in a row, the lower metal contacts of the elementary lasers being connected to a lower electrical track (CBMT) common to said elementary lasers of the row.

5. The medical probe according to claim 1, wherein said plurality of elementary lasers are disposed in a matrix, the lower semiconductor contacts of the elementary lasers of a row of the matrix being connected to a lower electrical track common to the elementary lasers of said row of the matrix, the lower electrical tracks associated with the rows being connected to each other.

6. The medical probe according to claim 1, wherein the lower and upper semiconductor contacts of an elementary laser are made of gallium nitride (GaN) or of a ternary material comprising gallium nitride.

7. The medical probe according to claim 6, wherein the two-dimensional material is graphene and the lower semiconductor contact has a crystallographic growth axis along the axis.

8. The medical probe according to claim 6, wherein the two-dimensional material is a dichalcogenide chosen from WS₂, MoS₂, ReS₂, and the lower semiconductor contact has a crystallographic growth axis along the axis.

9. The medical probe according to claim 1, wherein the lower and upper semiconductor contacts of an elementary laser are made of gallium arsenide (GaAs) or of a ternary material comprising gallium arsenide.

10. The medical probe according to claim 9, wherein the two-dimensional material is graphene, and wherein the graphene substrate comprises seats in which the elementary lasers are disposed.

11. The medical probe according to claim 1, wherein the active layer comprises multiple quantum wells (MQW) or quantum dots (QD).

12. The medical probe according to claim 1, wherein the substrate of two-dimensional material and the elementary lasers form a first structure (S1), the probe comprising at least one second structure (S2) stacked on the first structure (S1), elementary lasers of the two structures being arranged so that elementary lasers (μVL2) of the second structure do not mask a beam emitted (ELB1) by elementary lasers (μVL1) of the first structure.

13. The medical probe according to claim 12, wherein the elementary lasers in the second structure are configured to emit a wavelength (λ2) different from an emission wavelength (λ1) of the first structure.

14. The medical probe according to claim 13, wherein one of the two structures consists of elementary lasers comprising at least one layer of gallium nitride or a ternary material comprising gallium nitride, and the other structure consists of elementary lasers comprising at least one layer of gallium arsenide or a ternary material comprising gallium nitride.

15. A method for manufacturing a medical probe for optogenetics, comprising the steps of:
A1 making available a first initial substrate (IS1) comprising a semiconductor substrate (SS), an insulating layer substrate (ILS), a metal layer (ML), and a graphene layer (GS) disposed on the metal layer,
B1 depositing on the first initial substrate a first dielectric layer (DL1) and a first resin layer (RL1), structuring the first resin layer so as to form a first mask (M1) having first openings (Op1), the first openings having a maximum dimension of between 5 and 50 μm,
C1 etching the first dielectric layer as far as the graphene layer by wet etching, so as to expose the graphene layer in the first openings,
D1 removing the first resin layer,
E1 producing a stack of semiconductor materials in said first openings (Op1) by epitaxy, the stack comprising a lower semiconductor contact (BSCC) of gallium nitride epitaxially grown on the graphene layer, a lower reflective layer (BBR), an active layer (AL), an upper reflective layer (TBR), and an upper semiconductor contact (TSCC) made of gallium nitride,
F1 depositing a second dielectric layer (DL2), structuring a second resin layer (RL2) so as to form a second mask (M2) having second openings (Op2) above each stack, etching the second dielectric layer and the upper semiconductor contact, and removing the second resin layer,
G1 removing the first and second dielectric layers by wet etching, so as to obtain stacks disposed on the first initial substrate,
H1 depositing a lower metal contact (BMC) on the graphene layer, depositing an insulating layer around the stacks, and depositing an upper metal contact (TMC) connected to the upper semiconductor contact (TSCC),
I1 removing the semiconductor substrate (SS) using the insulating layer substrate (ILS),
J1 removing the metal layer (ML), the stacks and the associated first and second metal contacts being configured to form vertical-cavity surface-emitting semiconductor microlasers (μVL) inserted in the insulating layer, referred to as elementary lasers, the graphene layer forming a flexible substrate (GS), the elementary lasers being disposed on said flexible substrate, the method further comprising a step K1 of encapsulating the substrate and the elementary lasers with a biocompatible material.

16. The method according to claim 15, wherein, during step E1, the epitaxial growth of the lower gallium nitride semiconductor contact on the graphene substrate takes place in a direction.

17. The method according to claim 15, wherein, in step C1, the wet etching of the first dielectric layer as far as the graphene layer is of the buffered oxide etching (BOE) type.

18. The method for manufacturing a medical probe for optogenetics, comprising the steps of:
A2 making available a second initial substrate (IS2) comprising a gallium arsenide substrate (GAS), an insulating layer substrate (ILS), a metal layer (ML), and a first graphene layer (GL1) disposed on the metal layer,
B2 depositing on the second initial substrate a first dielectric layer (DL1) and a first resin layer (RL1), structuring the first resin layer so as to form a first mask (M1) having first openings (Op1), the first openings having a maximum dimension of between 5 and 50 82 m,
C2 etching the first dielectric layer as far as the graphene layer,
D2 removing the first resin layer, etching the graphene layer, etching the metal layer and etching the insulating layer substrate, so as to expose the gallium arsenide substrate in the first openings,
E2 producing by epitaxy, in said first openings (Op1), a stack of semiconductor materials comprising a lower semiconductor contact (BSCC) of gallium arsenide epitaxially grown on the gallium arsenide substrate, a lower reflective layer (BBR), an active layer (AL), an upper reflective layer (TBR), and an upper semiconductor contact (TSCC) made of gallium arsenide,
F2 depositing a second dielectric layer (DL2) and a second resin layer (RL2), structuring the second resin layer (RL2) so as to form a second mask (M2) having second openings (Op2) above each stack, etching the second dielectric layer, etching the upper semiconductor contact, and removing the second resin layer,
G2 removing the first and second dielectric layers by wet etching so as to obtain stacks disposed on the gallium arsenide substrate,
H2 depositing a lower metal contact (BMC) on the remaining graphene layer on each side of the stacks, depositing an insulating layer around the stacks, and depositing an upper metal contact (TMC) in contact with the upper semiconductor contact (TSCC),
I2 removing the gallium arsenide substrate (GAS) using the insulating layer substrate (ILS),
J2 removing the metal layer (ML), and depositing a second graphene layer (GL2) on the first graphene layer and the lower semiconductor contact, the first and second graphene layers collectively forming a flexible graphene substrate (GS), the stacks and the associated first and second metal contacts being configured to form vertical-cavity surface-emitting semiconductor microlasers (μVL) inserted in the insulating layer, referred to as elementary lasers, the elementary lasers being disposed on said graphene substrate, the graphene substrate comprising seats in which the elementary lasers are disposed, the method further comprising a step K2 of encapsulating the substrate and the elementary lasers with a biocompatible material.
